(19) United States
(12) Patent Application Publication
(10) Pub. No.: US 2021/0022832 A1
(43) Pub. Date: Jan. 28, 2021

(54) SYSTEMS AND METHODS FOR ORTHODONTIC DECISION SUPPORT

(71) Applicant: SmileDirectClub LLC, Nashville, TN (US)

(72) Inventors: Christopher Yancey, Nashville, TN (US); Ryan Ogletree, Nashville, TN (US)

(21) Appl. No.: 16/792,549

(22) Filed: Feb. 17, 2020

Related U.S. Application Data
(60) Provisional application No. 62/879,222, filed on Jul. 26, 2019.

(51) Int. Cl. A61C 7/00; A61C 9/00; G16H 50/50; G16H 10/60; G16H 20/30; G16H 70/20; G06T 7/73

(52) U.S. Cl. CPC A61C 7/002; A61C 9/0046; G16H 50/50; G16H 10/60; G06T 2207/30036; G16H 70/20; G06T 7/75; G06T 2207/20016; G16H 20/30

(57) ABSTRACT

A system for generating a desired result of an orthodontic treatment plan may include an orthodontic decision support system and a scanning system. The orthodontic decision support system generates orthodontic data that details the final desired position and orientation of the teeth of a user as part of a treatment plan for the user. The scanning system may gather orthodontic data for the user to input to the orthodontic decision support system in order to generate the final output.

### CROSS-REFERENCE TO RELATED PATENT APPLICATIONS

This application claims priority to U.S. Provisional Patent Application No. 62/879,222, filed Jul. 26, 2019, which is hereby incorporated by reference in its entirety.

### BACKGROUND

Dental aligners for repositioning a user's teeth may be manufactured for the user based on a 3D model of the user's teeth. The 3D model can be generated from a dental impression or an intraoral scan of the user's teeth. Dental impressions for generating such a 3D model can be taken by a user or an orthodontic professional using a dental impression kit. An intraoral scan of the user's mouth can be taken using 3D scanning equipment. Treatment plans using aligners may be created from the 3D model generated from a dental impression or an intraoral scan of the user's teeth.

### SUMMARY

An embodiment relates to a method. The method may include receiving an input comprising a 3D mesh of orthodontic data of a user including a position of axis parameter for each tooth of a plurality of teeth of the user, applying a metric model to the received input, and outputting a final position for each tooth based on applying the metric model to the received input. The metric model may use a metric comprising at least one of arch form data or tooth axes alignment data. In some implementations the metric model may use information relating to one or more of optimal occlusion (which may include information regarding molar interarch relationships, mesiodistal crown angulation, faciolingual inclination, labiolingual crown inclination, absence of rotations, tight contacts, curve of Spee, and Bolton's discrepency), ideal intercuspation, ideal overjet, ideal overbite, and pleasing profile. In some implementations, applying the metric model to the received input comprises varying the position of an axis parameter for one or more teeth to minimize the metric. In some implementations, the 3D mesh of orthodontic data of the user is segmented into data for each respective tooth. In some implementations, outputting a final position for each tooth further comprises outputting a delta from a current position of each tooth to the final position of each tooth. In some implementations, the input further include at least one of data from historical customer treatment plans or general rules and guidelines for positioning of teeth. In some implementations, the 3D mesh of orthodontic data of the user further includes root data for each tooth. In some implementations, the root data is from a scan of the user's teeth. In some implementations, the root data comprises virtual roots and the virtual roots are computed predictions of root locations based on the orthodontic data of the user. In some implementations, the root data comprises predicted roots and the predicted roots are generated by matching the orthodontic data of the user to a database comprising orthodontic data associated with respective root scans.

In some embodiments, the method further comprises modeling a periodontal ligament using the orthodontic data of the user, wherein the output of the final position for each tooth is further based on the modeling of the periodontal ligament. In some implementations, the method further comprises generating a treatment plan using the final position for each tooth based on applying the metric model to the received inputs.

Another embodiment relates to a system. The system may include a processing circuit comprising a processor communicably coupled to a non-transitory computer readable medium. The processor may execute one or more of the methods as described above.

Another embodiment relates to non-transitory computer readable media that store instructions that, when executed on a processing circuit, execute one or more of the methods as described above.

### BRIEF DESCRIPTION OF THE DRAWINGS

FIG. 1 is a block diagram of a system for generating a desired result of an orthodontic treatment plan, according to an example embodiment.

FIG. 2 is an illustrated block diagram of a process for extracting gingiva and teeth arch information from a 3D model.

FIG. 3A is an illustration of a first example photograph of a user's mouth.

FIG. 3B is an illustration of a second example photograph of a user's mouth.

FIG. 3C is an illustration of a third example photograph of a user's mouth.

FIG. 4 is an illustrated block diagram for generating a desired result of an orthodontic treatment plan from inputs, according to an example embodiment.

FIG. 5 is a flow diagram for generating a desired result of an orthodontic treatment plan, according to an example embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Described herein are systems and methods for orthodontic decision support. Where previously an individual, whether an orthodontic professional or not, may have to make a subjective determination of a final position of each tooth, the systems and methods disclosed herein allow for a computing device to make the determination. The final position and orientation of each tooth may be required to create a treatment plan for repositioning a user's teeth, for example using a system of molded aligners customized to the user, with a final goal of reaching as closely as possible a planned final position and orientation of each tooth. Instead of manually moving and repositioning each tooth in order to create a subjectively pleasing arch form and relative tooth orientation and tilt, the systems and methods for orthodontic decision support may be used to create the final position and orientation of each tooth. In some implementations, the systems and methods for orthodontic decision support may also be used to create intermediate positions and orientations of each tooth as intermediate goals to help minimize mid-course corrections in treatment.

Previous to the instant solution, an individual, whether an orthodontic professional or not, would have to make a subjective determination of a final position of each tooth prior to creating a treatment plan for each position. The individual would have to manually move teeth in a computer model in order to create a pleasing arch form and relative tooth orientation and tilt. Implementations of the orthodontic decision support systems and methods disclosed herein may solve the techno-centric problem of replacing this subjective determination with an automated determination by applying rules and/or a series of steps that allows a computing system to achieve results more efficiently or in a formulaic manner, unlike those previously achieved manually by individuals. In some implementations, the solution uses rules, rather than individuals (e.g., orthodontic professionals) to set weights and transitions between an initial position of a plurality of teeth and a final position of the plurality of teeth. For example, each respective user data from historical treatment plan data may be assigned a grade or score on the success of the final teeth positioning and orientation. Use of the historical treatment plan data in application of the model may weight portions of the data using these grades or scores.

In some implementations, the solution allows for a final position of arch form and relative tooth orientation and tilt that previously could only be accomplished by individuals (e.g., orthodontic professionals).

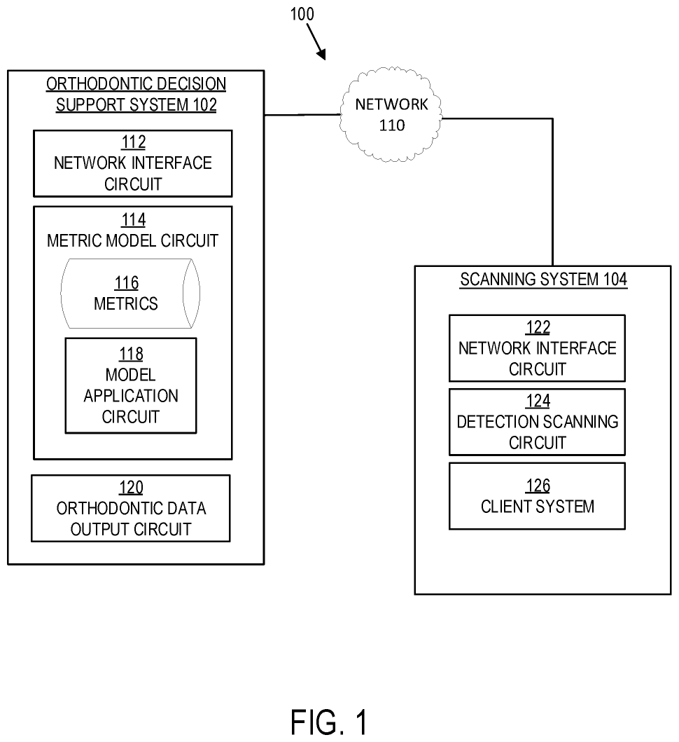

Referring now to FIG. 1, a system 100 for generating a desired result of an orthodontic treatment plan is shown according to an example illustration. The system 100 is shown to include an orthodontic decision support system 102 and a scanning system 104. As described in greater detail below, the orthodontic decision support system 102 may generate orthodontic data that details the final desired position and orientation of the teeth of a user as part of a treatment plan for the user. The scanning system 104 may gather orthodontic data for the user to input to the orthodontic decision support system 102 in order to generate the final output.

The system 100 is shown to include an orthodontic decision support system 102. The orthodontic decision support system 102 may include a network interface circuit 112, a metric model circuit 114, and an orthodontic data output circuit 120. The metric model circuit 114 may further comprise metrics 116 and a model application circuit 118. In some implementations, the network interface circuit 112 comprises one or more Bluetooth® transceivers, RFID transceivers, NFC transceivers, Wi-Fi transceivers, cellular transceivers, and the like. In some implementations, metric model circuit 114 and/or the orthodontic data output circuit 120 reside in part on different computing devices or systems (e.g., as part of the scanning system 104) in relation to other components or to the whole of a particular component. Data passing through the network interface circuit 112 may be encrypted such that the network interface circuit 112 is a secure communication module. In some arrangements, the network interface circuit 112, metric model circuit 114 (e.g., metrics 116 and model application circuit 118), and/or orthodontic data output circuit 120 reside in part on different servers in relation to other components or to the whole of a particular component.

In some implementations, the metric model circuit 114 is configured to apply a metric model to received orthodontic data. The metrics 116 may be configured as a database of metrics to use as part of the metric model to be applied to the received orthodontic data. Metrics may include inputs on what objectively makes a good smile (e.g., position of each tooth and how they relate to each other, arch form, axis alignment between respective teeth, tilt of each tooth, etc.). The model application circuit 118 of the metric model circuit 114 may be configured to receive orthodontic data via the network interface circuit 112. In some implementations, the orthodontic data includes at least a position of axis parameter for each tooth. In some implementations, the orthodontic data comprises a 3D mesh of orthodontic data from a user or patient. The 3D mesh of orthodontic data may have been obtained through an intraoral scan of the mouth of the user or from a scan of a received dental impression of the user or similar means. In some implementations the 3D mesh may include a computer generated mesh for each individual tooth. The 3D mesh may include data on features of each tooth, for example bumps, ridges, etc. In some implementations, the 3D mesh is used to create a 3D model of the teeth. In some implementations, a full 3D image of the teeth obtained through an intraoral scan of the mouth of the user or from a scan of a received dental impression of the user or similar means (e.g., using detection scanning circuit 124) can be used to generate a full computer generated mesh of the teeth, which can be used to generate a full 3D model of the teeth. In some implementations, the orthodontic data is the dimensional representation of the geometry of a dental impression, which is in turn a negative representation of a dental arch (e.g., a mandibular arch or maxillary arch) of a user. The model application circuit 118 may be configured to utilize STL files that describe the surface geometry of the corresponding dental impressions and include geometric faces which form a mesh which defines the surface geometry or contours. In other implementations, the data may be embodied as any surface or solid three-dimensional modeling data.

In some implementations, the model application circuit 118 is configured to apply a metric model to the received data (e.g., 3D mesh data). In some implementations, the metric model comprises metrics that are minimized in order to determine that a final desired output is reached. The model application circuit 118 may be configured to determine results for a final desired output that may comprise a desired teeth arch as well as final positions, orientations, and tilt of each respective tooth of the user. In some implementations, the metric model may use metrics that comprise at least one of arch form data or tooth axes alignment data. In some implementations, model application circuit 118 is configured to utilize the 3D mesh of orthodontic data of the user segmented into data for each respective tooth. In order to minimize the metrics, model application circuit 118 may be further configured to incorporate data from historical treatment plans and/or general rules and guidelines for the desired teeth arch as well as final positions, orientations and tilt of teeth with respect to the desired teeth arch and gingiva. In some implementations, the model application circuit 118 is configured to vary parameters (e.g., positions of axes for individual teeth) in order to minimize the metric.

In some implementations, the model application circuit 118 is further configured to determine how soft tissue is impacted by the orthodontic treatment and incorporates the determination into the determination of the desired teeth arch as well as tooth position. In some implementations, the model application circuit 118 is configured to determine how teeth roots are affected. The 3D mesh of orthodontic data may further include root data for each tooth. In some implementations, the root data is from a scan (e.g., an x-ray scan) of the user or patient. In some implementations, the root data comprises virtual roots, wherein the virtual roots are computed predictions of root locations based on the orthodontic data of the patient. In some implementations, the root data comprises predicted roots, wherein the orthodontic data of the patient may be compared to a database of orthodontic data indexed with scanned root data to find the closest match. In some implementations, a periodontal ligament of the user or patient may also be modeled using the orthodontic data of the patient. The additional root data and/or periodontal ligament data for the user may be incorporated in determining generating the final output position for each tooth.

In some implementations, the model application circuit 118 is configured to determine whether there is sufficient data to apply the metric model. In some implementations, a failure to receive an output may be indicative that insufficient, incomplete, and/or incorrect orthodontic data was received. In some implementations, a failure to receive an output that falls within a range of acceptable results may be indicative that insufficient, incomplete, and/or incorrect orthodontic data was received. If a determination is made there is insufficient data to apply the metric model, the model application circuit 118 may be configured to prompt for additional or replacement orthodontic data. In some cases, a new intraoral scan of the user or scan of a new dental impression may have to be conducted. In some instances, the model application circuit 118 may be configured to determine there is insufficient data prior to application of the metric model. In some implementations, a technician reviews the output of the model application circuit 118 to determine if insufficient, incomplete, and/or incorrect orthodontic data was received. The technician may, in some instances, be able to make some changes or adjustments manually. In some implementations, a technician may be able to make some changes or adjustments manually without needing additional or new data to apply the metric model.

In some implementations, the orthodontic data output circuit 120 is configured to output the final position for each tooth. In some implementations, the output is determined by application of the metric model to the received orthodontic data. In some implementations, the output is determined by minimizing the metrics of the metric model by varying parameters (e.g., positions of axes for individual teeth) in order to minimize the metric. The final desired output may comprise final positions, orientations, and tilt of each respective tooth of the user. In some implementations, the final desired output further comprises a desired teeth arch (e.g., a final top teeth arch and a final bottom teeth arch). In some implementations, the orthodontic data output circuit 120 is configured to output a delta from a current position of each tooth to the final position of each tooth. In some implementations, the orthodontic data output circuit 120 is configured to output a determination of how soft tissue is impacted by the orthodontic treatment. In some implementations, the orthodontic data output circuit 120 is configured to output a determination on how teeth roots are affected. In some implementations, the orthodontic data output circuit 120 is configured to output a determination on how a periodontal ligament of the user or patient is affected.

The system 100 is shown to include a scanning system 104. The orthodontic decision support system 102 may include a network interface circuit 122, a detection scanning circuit 124, and a client system 126. In some implementations, the network interface circuit 122 comprises one or more Bluetooth® transceivers, RFID transceivers, NFC transceivers, Wi-Fi transceivers, cellular transceivers, and the like. In some implementations, detection scanning circuit 124 and/or client system 126 reside in part on different computing devices or systems (e.g., as part of the orthodontic decision support system 102) in relation to other components or to the whole of a particular component. Data passing through the network interface circuit 122 may be encrypted such that the network interface circuit 122 is a secure communication module. In some arrangements, the network interface circuit 122, detection scanning circuit 124 and/or client system 126 reside in part on different servers in relation to other components or to the whole of a particular component.

In some implementations, the detection scanning circuit 124 is configured to conduct a scan of one or more objects. In this regard, the scanning circuit 124 may gather images of the object(s) being scanned (e.g., the size, shape, color, depth, tracking distance, and other physical characteristics) such that the data can be provided to other circuits in the system 100. To appropriately scan the target objects, the scanning circuit 124 can include a wide variety of sensors including, but not limited to, gyroscopes, accelerometers, magnetometers, inertial measurement units (“IMU”), depth sensors, and color sensors. In some implementations, the detection scanning circuit includes any device, component, or group of devices or components configured to generate dentition scans depicting the tooth and/or gingiva anatomy (referred to hereinafter as a “dental profile”) of a user or patient. In some embodiments, the dentition scans are digital scans of a physical dental impression, where the physical dental impression is captured by a dental technician, a dentist, or a user of a dental aligner. In some implementations, the scan (either of the patient's dentition or of the impression) is taken by the patient. The dentition scans may be direct scans of a user or patient's dentition. Hence, the dentition scans may be direct scans of a user or patient's dentition captured by scanning the user or patient's dentition with a three-dimensional camera, or the dentition scans may be indirect scans of the user or patient's dentition captured by scanning a physical model or impression of the user or patient's dentition. In either implementation, the dentition scans may be three-dimensional representations of a user or patient's dentition. The dentition scans may be used for generating a final positioning in a treatment plan for the user or patient.

In some implementations, the client system 126 is configured to receive dentition scans from the detection scanning circuit 124 and store the results associated with a user or patient. The client system 126 may be any number of different types of user electronic devices adapted to communicate via a network interface 122 and network 110, including without limitation, a personal computer, a laptop computer, a desktop computer, a mobile computer, a tablet computer, a smartphone, a dentition scanning system, or any other type and form of computing device or combinations of devices. The client system 126 may include a user application (e.g., a web browser) to facilitate the sending and receiving of data over the communication network 110.

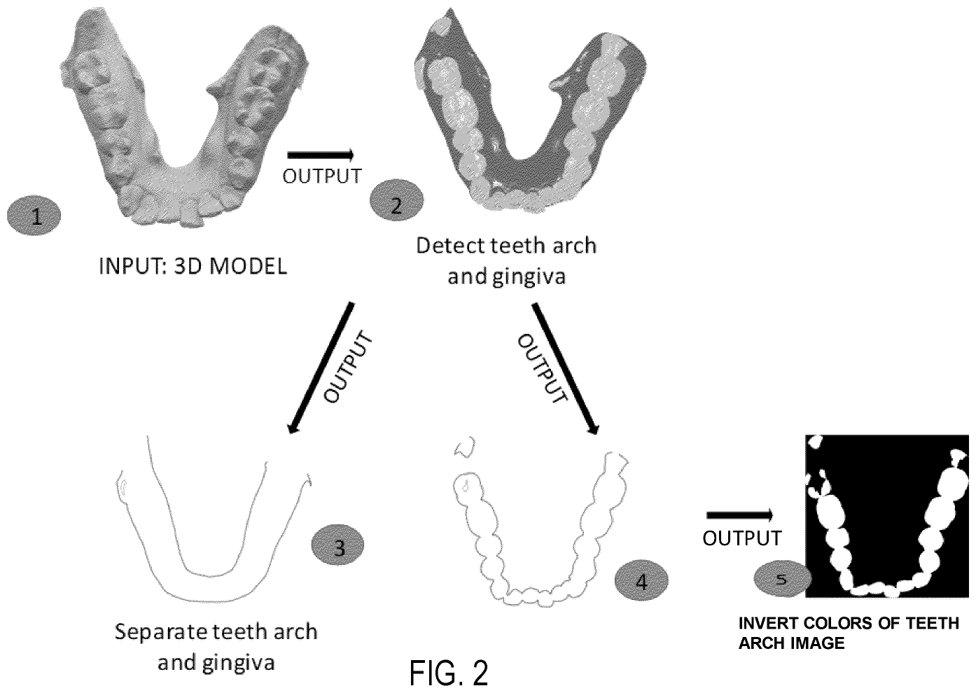

Referring now to FIG. 2, a block diagram of a process 200 for extracting gingiva and teeth arch information from a 3D model is illustrated according to an example embodiment. At step (1), a 3D model of a user or patient's teeth and gingiva are received. At step (2), the teeth arch and gingiva are detected using the 3D model. At steps (3) and (4), the teeth arch data and the gingiva data are separated. At step (5), the colors of the teeth arch image are inverted. In some implementations, a deep learning automated segmentation model is used that labels each point in the 3D model individually as gingiva or by tooth number.

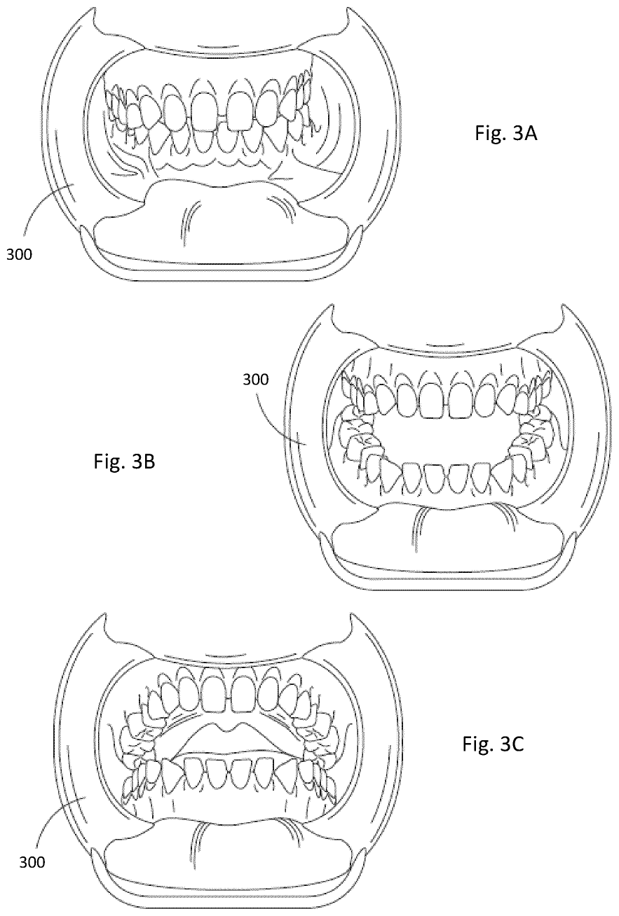

Referring now to FIGS. 3A-3C, example illustrations of photographs 300 of a user or patient's mouth are shown. For example, a view of a front smile in a closed, open, and upper open position is illustrated. Other views, such as a side view (not shown) may be use. These views may be analyzed to extract features and merge with labeled data from chief complaint of a user or patient and validating. The analyzed views may be used for detecting types of malocclusion. In some implementations, photographs (e.g., photographs such as those illustrated by photographs 300) of a user are used as part of the process of orthodontic decision support and may be used to determine whether the patient qualifies for treatment. In some implementations, photographs (e.g., photographs such as those illustrated by photographs 300) of a user are used to inform the final position of a plurality of teeth of a user. In some implementations, the patient may take these photograph(s) themselves in the form of a “selfie” or have others help take these photographs. As referred to herein, a “selfie” may be a photo taken of oneself using the front-facing camera of a device equipped with such a camera. In some implementations, the patient may take these photograph(s) themselves in the form of a selfie or have others help take these photographs as part of an at-home impression process. In some implementations, a dental technician or orthodontic professional may take these photograph(s) as part of an intraoral scan appointment.

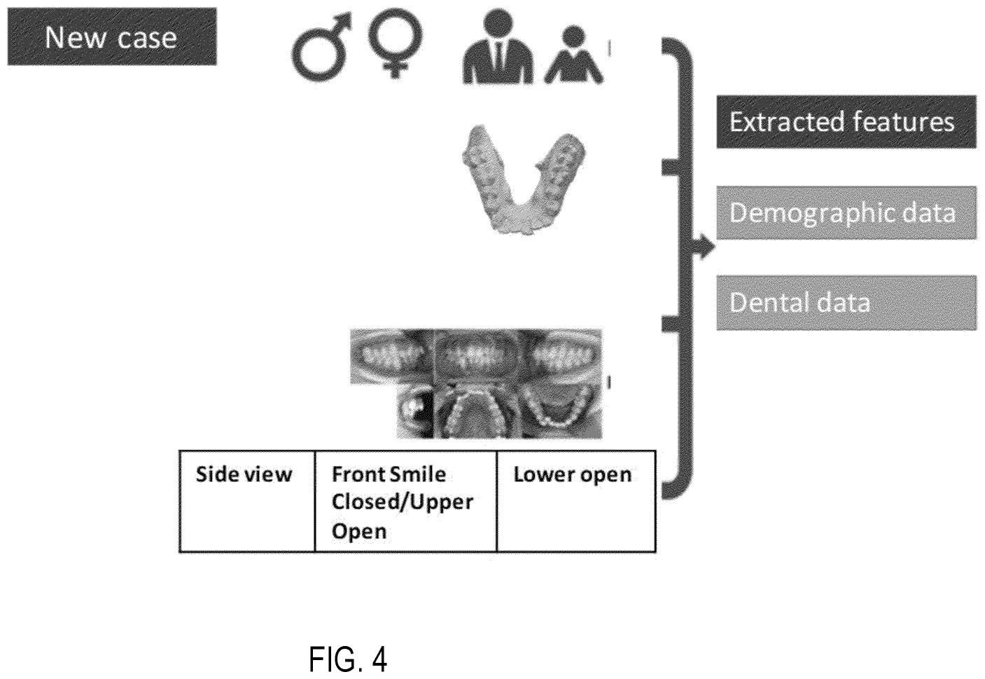

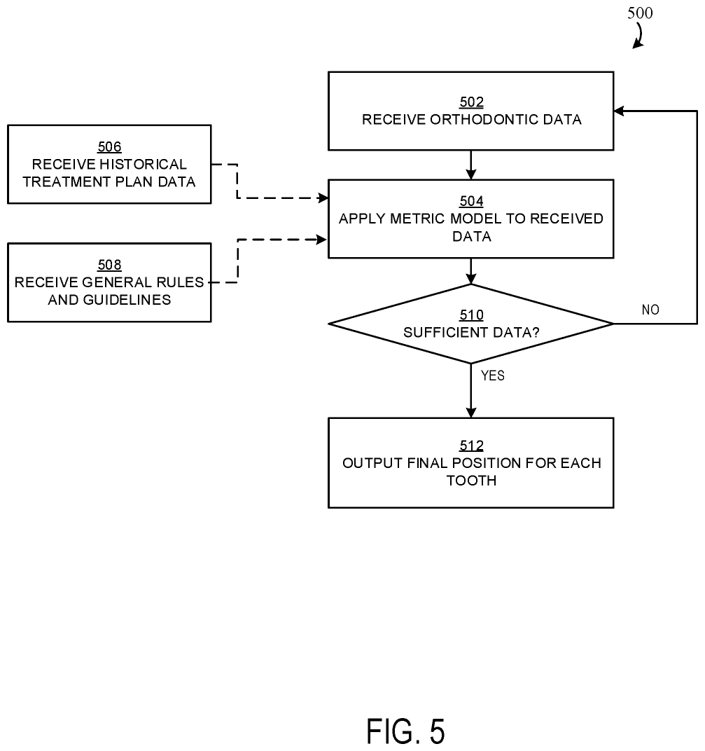

Referring now to FIG. 4, an illustrated block diagram for generating a desired result of an orthodontic treatment plan from inputs is shown according to an example implementation. In some implementations, a metric model may include data from extracted features of a user or patient from photographs (e.g., example illustrations of photographs 300), demographic data of the user or patient, and orthodontic data of the user or patient to create a desired result of an orthodontic treatment plan as discussed further below in reference to FIG. 5. The analysis of the inputted data may determine categorization of a new case into predefined classes, identification of a type of malocclusion for the new case, and recommendation for the best treatment plan for the user or patient.

Referring now to FIG. 5, a flow diagram of a method 500 for generating a desired result of an orthodontic treatment plan is shown according to an example implementation. The method 500 may be implemented by one or more of the components described above, for example by the orthodontic decision support system 102. As an overview, the method may comprise, receiving orthodontic data at 502, applying a metric model to the received orthodontic data at 504, and determining whether there is sufficient data to apply the model at 510. If a determination is made that there is sufficient data to apply the metric model, the method may further comprise outputting the final position for each tooth at 512. If a determination is made there is insufficient data to apply the model, the method may return to 502 to receive replacement or additional orthodontic data. In addition, the method may further comprise receiving historical treatment plan data at 506 and/or receiving general rules and guidelines at 508.

Still referring to FIG. 5, and in more detail, orthodontic data is received at 502. In some implementations, the orthodontic data includes at least a position of axis parameter for each tooth. In some implementations, the orthodontic data comprises a 3D mesh of orthodontic data from a user or patient. The 3D mesh of orthodontic data may have been obtained through an intraoral scan of the mouth of the user or from a scan of a received dental impression of the user or similar means. In some implementations the 3D mesh may include a computer generated mesh for each individual tooth. The 3D mesh may include data on features of each tooth, for example bumps, ridges, etc. In some implementations, the 3D mesh is used to create a 3D model of the teeth. In some implementations, a full 3D image of the teeth obtained through an intraoral scan of the mouth of the user or from a scan of a received dental impression of the user or similar means can be used to generate a full computer generated mesh of the teeth, which can be used to generate a full 3D model of the teeth. In some implementations, the orthodontic data is the dimensional representation of the geometry of a dental impression, which is in turn a negative representation of a dental arch (e.g., a mandibular arch or maxillary arch) of a user. These may be embodied as STL files that describe the surface geometry of the corresponding dental impressions and include geometric faces which form a mesh which defines the surface geometry or contours. In other implementations, the data may be embodied as any surface or solid three-dimensional modeling data.

A metric model is applied to the received data at 504. In some implementations, the metric model comprises metrics that are minimized in order to determine that a final desired output is reached. The final desired output may comprise a desired teeth arch as well as final positions, orientations, and tilt of each respective tooth of the user. In some implementations, the metric model may use metrics that comprise at least one of arch form data or tooth axes alignment data. Other non-limiting parameters that may form the metric include gaps between adjacent teeth, shape of a top arch of the user including length and width, shape of a bottom arch of a user including length or width, presence of an overbite including which teeth constitute the overbite and a numerical length and/or tilt measurement associated with the overbite, rotational angles of one or more of the teeth of the user with respect to the front of the user's mouth, a tilt of one or more teeth of the user with respect to the front of the mouth and/or other reference point associated with the user's mouth, symmetry of the teeth, and the like. In some implementations, the 3D mesh of orthodontic data of the user is segmented into data for each respective tooth. In order to minimize the metrics, the metric model may also incorporate data from historical treatment plans and/or general rules and guidelines for desired teeth arch as well as final positions, orientations and tilt of teeth with respect to the desired teeth arch and gingiva. In some implementations, application of the metric model comprises varying parameters (e.g., positions of axes for individual teeth) in order to minimize the metric. In some implementations, least absolute deviations (“L1”) and/or least square errors (“L2”) loss functions are used. The loss functions may determine what function should be minimized while learning from the dataset. Customized losses may be used for particular portions of the metric model. For example, a customized signed distance function that is non symmetric about the ‘0’ may be used for occlusal contacts. In some implementations, minimizing the metric may include minimizing Root Mean Square (RMS) deviation. In some implementations, minimizing the metric may include minimizing the Mean Absolute Error (MAE). In some implementations, minimizing the metric may include minimizing the Mean Signed Difference (MAD). In some implementations, minimizing the metric may include minimizing the Mean Squared Error (MAE). For example, varying the parameters may include altering the axes of adjacent teeth to reduce a space between the tooth. In another example, varying the parameters may include altering the axes of teeth to alter a curve of a top arch or curve of a bottom arch. In a further example, varying the parameters may include altering the tilt of teeth in order to minimize an overbite.

In some implementations, a determination is made on how soft tissue (e.g., the lips, the gums or gingiva, etc.) is impacted by the orthodontic treatment and is incorporated into the determination of the desired teeth arch as well as tooth position. For example, the shape of smiles may be affected by soft tissue such as the lip line or the height of the upper live relative to the maxillary central incisors, the upper lip length and the relationship of the upper lip to the maxillary incisors, the lip elevation and amount of maxillary incisors displayed, the vertical maxillary height displayed, crown height as affected by gingival encroachment, vertical dental height, smile arc, upper lip curvature, smile symmetry, color contour, texture, and height of gingivae, and the like. In some implementations, a determination is made on how teeth roots are affected. For example, a determination of an expected amount, if any, of root damage, root loss and/or root resorption. The 3D mesh of orthodontic data may further include root data for each tooth. In some implementations, the root data is from a scan (e.g., an x-ray scan) of the user or patient. In some implementations, the root data comprises virtual roots, wherein the virtual roots are computed predictions of root locations based on the orthodontic data of the patient. In some implementations, the root data comprises predicted roots, wherein the orthodontic data of the patient may be compared to a database of orthodontic data indexed with scanned root data to find the closest match. In some implementations, a periodontal ligament of the user or patient may also be modeled using the orthodontic data of the patient. The additional root data and/or periodontal ligament data for the user may be incorporated in determining generating the final output position for each tooth.

A determination of whether there is sufficient data to apply the metric model is made at 510. In some implementations, a failure to receive an output may be indicative that insufficient, incomplete, and/or incorrect orthodontic data was received. In some implementations, a failure to receive an output that falls within a range of acceptable results may be indicative that insufficient, incomplete, and/or incorrect orthodontic data was received. If a determination is made there is insufficient data to apply the metric model, the method may return to receiving orthodontic data at 502. In some cases, a new intraoral scan of the user or scan of a new dental impression or rescan of a dental impression may have to be conducted. In some instances, the determination that there is insufficient data to apply the metric model may be done prior to 504, e.g., before the metric model is applied to the received data. If a determination is made there is sufficient data and/or that the model has been successfully applied to the data, the method may continue to outputting the final position for each tooth at 512.

The final position for each tooth is output at 512. In some implementations, the output is determined by application of the metric model to the received orthodontic data. In some implementations, the output is determined by minimizing the metrics of the metric model by varying parameters (e.g., positions of axes for individual teeth) in order to minimize the metric. The final desired output may comprise final positions, orientations, and tilt of each respective tooth of the user. In some implementations, the orthodontic data output circuit 120 is configured to output a delta from a current position of each tooth to the final position of each tooth. In some implementations, the final desired output further comprises a desired teeth arch (e.g., a final top teeth arch and a final bottom teeth arch). In some implementations, a determination of how soft tissue is impacted by the orthodontic treatment is also outputted. In some implementations, a determination on how teeth roots are affected is also outputted. In some implementations, a determination on how a periodontal ligament of the user or patient is also outputted.

In some implementations, historical treatment plan data is received at 506. The historical treatment plan data may comprise data on actual users or patients including initial teeth positions and orientations along with final teeth positions and orientations after treatment. In some implementations, each respective user data may be assigned a grade or score on the success of the final teeth positioning and orientation. In some implementations, use of the historical treatment plan data in application of the model may weight portions of the data using these grades or scores.

In some implementations, general rules and guidelines are received at 508. In some implementations, the general rules and guidelines are used in the application of the model to incoming orthodontic data. The rules and guidelines may comprise a set of limits outside of which final tooth arch and tooth positioning data may not be set.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be X, Y, or Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

The hardware and data processing components used to implement the various processes, operations, illustrative logics, logical blocks, modules, and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, flash memory, hard disk storage) for storing data and/or computer code for completing or facilitating the various processes, layers and circuits described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data, which cause a general-purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems and methods shown in the various exemplary embodiments are illustrative only. Additionally, any element disclosed in one embodiment may be incorporated or utilized with any other embodiment disclosed herein.

1. A method comprising:
receiving, by a processor configured to carry out instructions stored on a communicably coupled non-transitory computer readable medium, an input comprising a 3D mesh of orthodontic data of a user including a position of axis parameter for each tooth of a plurality of teeth of the user;
applying, by the processor, a metric model to the received input, wherein the metric model uses a metric comprising at least one of arch form data or tooth axes alignment data;
determining, by the processor, whether the input satisfies a data sufficiency requirement for applying the metric model by determining that an output from applying the metric model falls within a predetermined range;
outputting, by the processor, based on determining that the input satisfies the data sufficiency requirement, a final position for each tooth based on applying the metric model to the received input.

2. The method of claim 1, wherein applying the metric model to the received input comprises varying the position of an axis parameter for each tooth to minimize the metric.

3. The method of claim 1, wherein the 3D mesh of orthodontic data of the user is segmented into data for each respective tooth.

4. The method of claim 1, wherein outputting the final position for each tooth further comprises outputting a delta from a current position of each tooth to the final position of each tooth.

5. The method of claim 1, wherein the input further includes data from historical customer treatment plans.

6. The method of claim 1, wherein the 3D mesh of orthodontic data of the user further includes root data for each tooth.

7. The method of claim 6, wherein (1) the root data is from a scan of the user's teeth or (2) the root data comprises virtual roots and the virtual roots are computed predictions of root locations based on the orthodontic data of the user.

8. The method of claim 6, wherein the root data comprises predicted roots, wherein the predicted roots are generated by matching the orthodontic data of the user to a database comprising orthodontic data associated with respective root scans.

9. The method of claim 1, further comprising modeling a periodontal ligament using the orthodontic data of the user, wherein the output of the final position for each tooth is further based on the modeling of the periodontal ligament.

10. The method of claim 1, further comprising generating a treatment plan using the final position for each tooth based on applying the metric model to the received input.

11. A system comprising:
a processing circuit comprising a processor communicably coupled to a non-transitory computer readable medium, wherein the processor is configured to execute instructions stored on the non-transitory computer readable medium to cause the processor to:
receive an input comprising a 3D mesh of orthodontic data of a user including at least a position of axis parameter for each tooth of a plurality of teeth of the user;
apply a metric model to the received input, wherein the metric model uses a metric comprising at least one of arch form data or tooth axes alignment data;
determine whether the input satisfies a data sufficiency requirement for applying the metric model by determining that an output from applying the metric model falls within a predetermined range;
output, based on a determination that the input satisfies the data sufficiency requirement, a final position for each tooth based on applying the metric model to the received input.

12. The system of claim 11, wherein application of the metric model to the received input comprises varying the position of an axis parameter for each tooth to minimize the metric.

13. The system of claim 11, wherein the 3D mesh of orthodontic data of the user is segmented into data for each respective tooth.

14. The system of claim 11, wherein outputting the final position for each tooth further comprises outputting a delta from a current position of each tooth to the final position of each tooth.

15. The system of claim 11, wherein the input further includes at least one of (1) data from historical customer treatment plans or (2) general rules and guidelines for positioning of teeth.

16. The system of claim 11, wherein the 3D mesh of orthodontic data of the user further includes root data for each tooth.

17. The system of claim 16, wherein the root data comprises at least one of a scan of the user's teeth, virtual roots that are computed predictions of root locations based on the orthodontic data of the user, or predicted roots that are generated by matching the orthodontic data of the user to a database comprising orthodontic data associated with respective root scans.

18. The system of claim 11, the processor further configured to model a periodontal ligament using the orthodontic data of the user, wherein the output of the final position for each tooth is further based on the modeling of the periodontal ligament.

19. The system of claim 11, the processor further configured to generate a treatment plan using the final position for each tooth based on applying the metric model to the received input.

20. A non-transitory computer-readable storage media storing instructions that are executable by one or more processors to perform operations comprising:
receiving an input comprising a 3D mesh of orthodontic data of a user including a position of axis parameter for each tooth of a plurality of teeth of the user, wherein the 3D mesh of orthodontic data of the user is segmented into data for each respective tooth;
applying a metric model to the received input by varying the position of an axis parameter for each tooth to minimize a metric, wherein the metric model uses the metric comprising at least one of arch form data or tooth axes alignment data;
determining whether the input satisfies a data sufficiency requirement for applying the metric model by determining that an output from applying the metric model falls within a predetermined range;
outputting, based on determining that the input satisfies the data sufficiency requirement, a final position for each tooth based on applying the metric model to the received input including a delta from a current position of each tooth to the final position of each tooth; and
wherein the input further includes at least one of (1) data from historical customer treatment plans or (2) general rules and guidelines for positioning of teeth,
wherein the 3D mesh of orthodontic data of the user further includes root data for each tooth,
wherein (1) the root data is from a scan of the user's teeth, (2) the root data comprises virtual roots and the virtual roots are computed predictions of root locations based on the orthodontic data of the user, or (3) the root data comprises predicted roots, wherein the predicted roots are generated by matching the orthodontic data of the user to a database comprising orthodontic data associated with respective root scans.

21. The method of claim 10, wherein the generated treatment plan is used to manufacture a plurality of dental aligners configured to reposition teeth of the user based on the final position for each tooth.

22. The system of claim 19, wherein the generated treatment plan is used to manufacture a plurality of dental aligners configured to reposition teeth of the user according to the final position for each tooth.

23. The non-transitory computer-readable storage media of claim 20, wherein the operations further comprise generating a treatment plan using the final position for each tooth, wherein the generated treatment plan is used to manufacture a plurality of dental aligners configured to reposition teeth of the user based on the final position for each tooth.
